(19) United States
(12) Patent Application Publication
(10) Pub. No.: US 2022/0331507 A1
(43) Pub. Date: Oct. 20, 2022

(54) SYSTEMS AND METHODS FOR POST-OCCLUSION BREAK SURGE MITIGATION

(71) Applicant: Alcon Inc., Fribourg (CH)

(72) Inventors: Vincent A. Baxter, Temecula, CA (US); David Dyk, Foothill Ranch, CA (US); Satish Yalamanchili, Irvine, CA (US); Guangyao Jia, Irvine, CA (US); Yen-Cheng Chen, Orange, CA (US)

(21) Appl. No.: 17/658,834

(22) Filed: Apr. 12, 2022

Related U.S. Application Data
(60) Provisional application No. 63/175,590, filed on Apr. 16, 2021.

(51) Int. Cl. A61M 1/00; A61F 9/007; A61F 9/008

(52) U.S. Cl. CPC A61M 1/77; A61F 9/00736; A61F 9/00745; A61F 9/008; A61M 1/74; A61F 2009/00887; A61F 2009/0087

(57) ABSTRACT

A surgical cassette for an ophthalmic surgical system includes an irrigation system, an aspiration system, and a computer. The irrigation system is in fluid communication with a handpiece and carries fluid toward a surgical site. The aspiration system is in fluid communication with the handpiece via an aspiration conduit and carries fluid away from the surgical site. The computer instructs the aspiration system to vibrate fluid back and forth within the aspiration conduit to perform a priming procedure.

### TECHNICAL FIELD

The present disclosure relates to ophthalmic surgical systems and methods, and more particularly to systems and methods that mitigate post-occlusion break surges during ophthalmic surgery.

### BACKGROUND

Cataract surgery involves removing the cataractous lens and replacing the lens with an artificial intraocular lens (IOL). The cataractous lens is typically removed by fragmenting the lens and aspirating the lens fragments out of the eye. The lens may be fragmented using, e.g., a phacoemulsification handpiece, a laser handpiece, or other suitable handpiece. During the procedure, the handpiece fragments the lens, and the fragments are aspirated out of the eye through, e.g., a hollow needle. Throughout the procedure, irrigating fluid is pumped into the eye to maintain an intraocular pressure (IOP) to prevent collapse of the eye.

A complication arises when there is a blockage, or occlusion, of the needle. As the irrigation fluid and emulsified tissue are aspirated through the hollow needle, pieces of tissue may clog the tip, and vacuum pressure builds up within the tip. An occlusion break occurs when the tissue breaks free and moves through the needle. When this happens, the vacuum pressure in the anterior chamber suddenly drops, resulting in a post-occlusion break surge. In some cases, the post-occlusion break surge can cause a relatively large quantity of fluid and tissue to be aspirated out of the eye too quickly, potentially collapsing the eye and/or tearing the lens capsule. Known techniques for mitigating this surge are not satisfactorily effective in certain situations.

### BRIEF SUMMARY

According to certain embodiments, a surgical cassette for an ophthalmic surgical system includes an irrigation system, an aspiration system, and a computer. The irrigation system is in fluid communication with a handpiece and carries fluid toward a surgical site. The aspiration system is in fluid communication with the handpiece via an aspiration conduit and carries fluid away from the surgical site. The computer instructs the aspiration system to vibrate fluid back and forth within the aspiration conduit to perform a priming procedure.

Embodiments may include none, one, some, or all of the following features: The aspiration system comprises a valve that vibrates fluid by opening to move fluid into the aspiration conduit, and closing to cease moving the fluid into the aspiration conduit. The aspiration system comprises an aspiration pump that creates a vacuum pressure within the aspiration conduit, and vibrates fluid by changing the vacuum pressure within the aspiration conduit. The aspiration conduit comprises tubing with an internal surface that is hydrophilic to facilitate removal of air bubbles within the tubing. The aspiration conduit comprises tubing with an internal surface that is superhydrophilic to facilitate removal of air bubbles within the tubing. The aspiration system comprises a fluidics mechanism that vibrates the surgical cassette to vibrate the fluid. The aspiration system comprises tubing that carries the fluid, and a fluidics mechanism that vibrates the tubing. The computer instructs the handpiece to run to vibrate the fluid. ccording to certain embodiments, a surgical cassette for an ophthalmic surgical system includes an irrigation system, an aspiration system, and a computer. The irrigation system is in fluid communication with a handpiece, and carries fluid toward a surgical site. The aspiration system is in fluid communication with the handpiece via an aspiration conduit, and carries fluid away from the surgical site. The computer instructs the handpiece to run to vibrate fluid back and forth within the aspiration conduit to perform a priming procedure.

Embodiments may include none, one, some, or all of the following features: The aspiration conduit comprises tubing with an internal surface that is hydrophilic to facilitate removal of air bubbles within the tubing. The aspiration conduit comprises tubing with an internal surface that is superhydrophilic to facilitate removal of air bubbles within the tubing. The computer instructs the aspiration system to vibrate fluid back and forth within the aspiration conduit. The aspiration system may comprise a fluidics mechanism that vibrates the surgical cassette to vibrate the fluid. The aspiration system may comprise tubing that carries the fluid, and a fluidics mechanism that vibrates the tubing.

According to certain embodiments, a method for performing a priming procedure for a surgical cassette comprises: carrying, by an irrigation system in fluid communication with a handpiece, fluid toward a surgical site; carrying, by an aspiration system in fluid communication with the handpiece via an aspiration conduit, fluid away from the surgical site; and instructing, by a computer, the aspiration system to vibrate fluid back and forth within the aspiration conduit.

Embodiments may include none, one, some, or all of the following features: The method further comprises: opening, by a valve of the aspiration system, to move fluid into the aspiration conduit; and closing, by the valve, to cease moving the fluid into the aspiration conduit. The method further comprises: creating, by an aspiration pump of the aspiration system, a vacuum pressure within the aspiration conduit; and vibrating, by the aspiration pump, fluid by changing the vacuum pressure within the aspiration conduit. The method further comprises: vibrating, by a fluidics mechanism of the aspiration system, the surgical cassette to vibrate the fluid. The method further comprises: vibrating, by a fluidics mechanism of the aspiration system, tubing of the aspiration system that carries the fluid in order to vibrate the fluid.

According to certain embodiments, a surgical cassette for an ophthalmic surgical system includes an irrigation system, an aspiration system, and a computer. The irrigation system is in fluid communication with a handpiece, and carries fluid toward a surgical site. The aspiration system is in fluid communication with the handpiece via an aspiration conduit, and carries fluid away from the surgical site. The computer instructs the aspiration system to vibrate fluid back and forth within the aspiration conduit to perform a priming procedure, and instructs the handpiece to run to vibrate the fluid. The aspiration system comprises: a valve that vibrates fluid by opening to move fluid into the aspiration conduit, and closing to cease moving the fluid into the aspiration conduit; an aspiration pump that creates a vacuum pressure within the aspiration conduit, and vibrates fluid by changing the vacuum pressure within the aspiration conduit; tubing with an internal surface that is hydrophilic to facilitate removal of air bubbles within the tubing; and a fluidics mechanism that vibrates the surgical cassette and the tubing to vibrate the fluid.

### BRIEF DESCRIPTION OF THE DRAWINGS

FIG. 1 illustrates an example of an ophthalmic surgical system that may be used to perform ophthalmic procedures on an eye, according to certain embodiments;

FIG. 2 is an example of subsystems of a console of the ophthalmic surgical system of FIG. 1, according to certain embodiments;

FIG. 3 illustrates an example of a fluidics subsystem that may be used with the surgical console of the ophthalmic surgical system of FIGS. 1 and 2, according to certain embodiments;

FIG. 4 illustrates an example of the fluidics subsystem of FIG. 3 that includes an example of a variable volume chamber that may mitigate post-occlusion break surges;

FIG. 5 illustrates an example of the handpiece of FIG. 3 that includes a variable volume chamber that may mitigate post-occlusion break surges;

FIG. 6 illustrates an example of a method that may be used by the fluidics subsystem of FIG. 3 to mitigate a post-occlusion break surge during a surgical procedure, according to certain embodiments;

FIG. 7 illustrates an example of the fluidics subsystem of FIG. 3 that includes an example of a chamber system that may mitigate post-occlusion break surges;

FIG. 8 illustrates an example of a method that may be used by the fluidics subsystem of FIG. 3 to mitigate a post-occlusion break surge during a surgical procedure, according to certain embodiments;

FIG. 9 illustrates an example of the fluidics subsystem of FIG. 3 that performs a priming procedure that may improve mitigation of post-occlusion break surges;

FIG. 10 illustrates an example of the fluidics subsystem of FIG. 3 that includes an example of an irrigation system that may mitigate post-occlusion break surges;

FIG. 11 illustrates an example of a method that may be used by the fluidics subsystem of FIG. 3 to mitigate a post-occlusion break surge during a surgical procedure, according to certain embodiments;

FIG. 12 illustrates an example of tubing that comprises a mitigating viscoelastic material;

FIGS. 13A and 13B illustrate an example of tubing with a mitigating cross-section; and

FIGS. 14A, 14B, and 14C illustrate an example of tubing with a mitigating section with a mitigating cross-section.

### DESCRIPTION OF EXAMPLE EMBODIMENTS

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately. For simplicity, in some instances the same reference numbers are used throughout the drawings to refer to the same or like parts.

The present disclosure relates generally to devices, systems, and methods for performing lens fragmentation procedures. During fragmentation, mitigating a post-occlusion break surge can be critical to the success of the procedure. The devices, system, and methods disclosed herein include features for mitigating post-occlusion break surges.

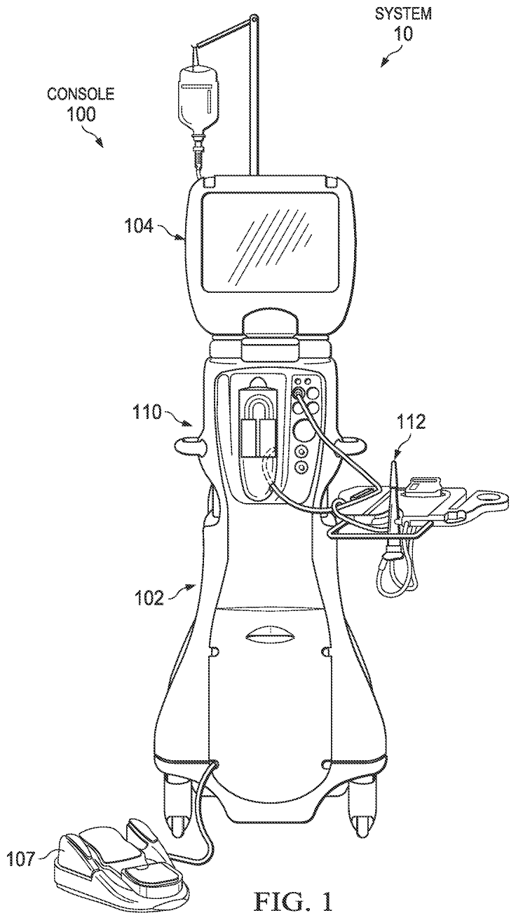

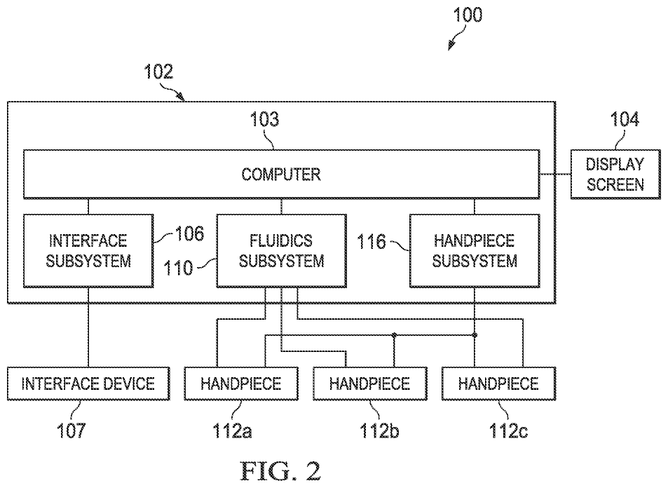

FIG. 1 illustrates an example of an ophthalmic surgical system 10 that may be used to perform ophthalmic procedures on an eye, according to certain embodiments. In the illustrated example, system 10 includes console 100, a housing 102, a display screen 104, an interface device 107 (e.g., a foot pedal), a fluidics subsystem 110, and a handpiece 112, coupled as shown and described in more detail with reference to FIG. 2.

FIG. 2 is an example of subsystems of console 100 of ophthalmic surgical system 10 of FIG. 1, according to certain embodiments. Console 100 includes housing 102, which accommodates a computer 103 (with an associated display screen 104) and subsystems 106, 110, and 116, which support interface device 107 and handpieces 112 (112a-c). An interface device 107 receives input to surgical system 10, sends output from system 10, and/or processes the input and/or output. Examples of an interface device 107 include a foot pedal, manual input device (e.g., a keyboard), and a display. Interface subsystem 106 receives input from and/or sends output to interface device 107.

Handpiece 112 may be any suitable ophthalmic surgical instrument, e.g., an ultrasonically-driven phacoemulsification (phaco) handpiece, a laser handpiece, an irrigating cannula, a vitrectomy handpiece, or other suitable surgical handpiece. Fluidics subsystem 110 provides fluid control for one or more handpieces 112 (112a-c). For example, fluidics subsystem 110 may manage fluid for an irrigating cannula. Handpiece subsystem 116 supports one or more handpieces 112. For example, handpiece subsystem 116 may manage ultrasonic oscillation for a phaco handpiece, provide laser energy to a laser handpiece, control operation of an irrigating cannula, and/or manage features of a vitrectomy handpiece.

Computer 103 controls operation of ophthalmic surgical system 10. In certain embodiments, computer 103 includes a controller that sends instructions to components of system 10 to control system 10. A display screen 104 shows data provided by computer 103.

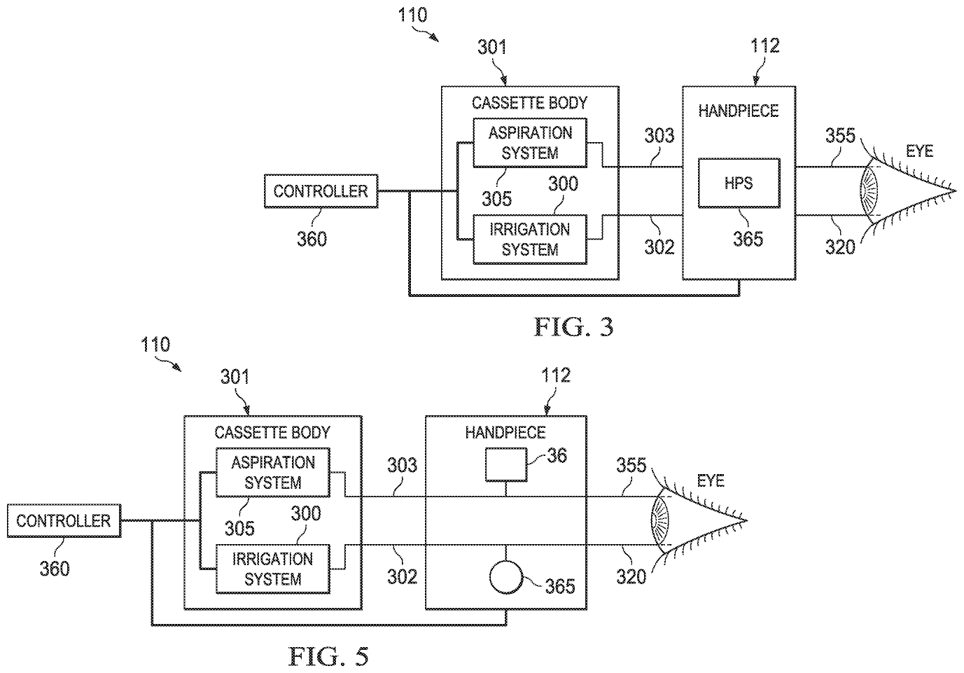

FIG. 3 illustrates an example of a fluidics subsystem 110 that may be used with surgical console 100 of ophthalmic surgical system 10 of FIGS. 1 and 2, according to certain embodiments. In general, a controller 360 (such as computer 103) controls parts of fluidics subsystem 110 to maintain a target intraocular pressure (IOP) of the eye (e.g., a value in the range of 0 to 110 millimeters of mercury (mmHg)) during a surgical procedure performed with handpiece 112. If controller 360 determines the IOP is outside of the target range, controller 360 controls fluidics subsystem 110 to bring the pressure back to the target range. For example, a post-occlusion break may create an unacceptable surge in volume demand from the eye. To mitigate the surge, the fluidics subsystem may meet the fluid demand before the volume is demanded from the eye.

In the illustrated example, fluidics subsystem 110 has a cassette body 301 that may be accommodated by surgical console 100 as a surgical cassette. Fluidics subsystem 110 includes an irrigation system 300 and an aspiration system 305, which are controlled by controller 360. Irrigation system 300 and aspiration system 305 are in fluid communication with a handpiece 112. During normal operation, irrigation system 300 carries fluid toward a surgical site, and aspiration system 305 carries fluid away from the surgical site. Irrigation conduit 302 provides fluid communication between irrigation system 300 and handpiece 112, and aspiration conduit 303 provides fluid communication between aspiration system 305 and handpiece 112. Parts that are in fluid communication with each other are parts for which fluid is allowed to flow between (to and/or from) the parts.

Aspiration system 305 carries fluid away from the surgical site by creating and maintaining a vacuum pressure (or negative pressure) in a vacuum path of an aspiration conduit 303. Vacuum pressure can be described as negative pressure. Accordingly, increasing the vacuum pressure may be described as increasing negative pressure or decreasing pressure, and decreasing the vacuum pressure may be described as decreasing negative pressure or increasing pressure. In certain embodiments, an aspiration pump of aspiration system 305 may be reversed to supply fluid to aspiration conduit 303 in order to mitigate a post-occlusion break surge.

Handpiece 112 includes an irrigation channel 320, an aspiration channel 355, and a handpiece pressure sensor (HPS) 365. Irrigation channel 320 provides fluid to the surgical site, and may be an irrigating tip or an irrigating sleeve that surrounds aspiration channel 355. Aspiration channel 355 may be a hollow needle that vibrates at a fixed frequency to break up tissue. Fluid and tissue may be aspirated through the needle. In certain embodiments, handpiece 112 may be an ultrasonically-driven phaco handpiece or a laser handpiece that uses laser energy to fragment the lens to facilitate the phacoemulsification process.

HPS 365 is an irrigation pressure sensor that detects the irrigation pressure within the irrigation conduit 302. In the illustrated example, HPS 365 is located on handpiece 112 close to the surgical site, e.g., less than 12 inches from the surgical site. The proximity to the surgical site may enable quick detection of changes in pressure (as may occur during an occlusion break) and allow for real-time surge suppression. In some examples, HPS 365 detects pressure changes within 50 milliseconds of an occlusion break, which may enable controller 360 to respond to pressure deviations before IOP is overly negatively affected. In general, an irrigation pressure sensor may be located at any suitable location, such as any suitable location of handpiece 112 (e.g., the proximal end, the distal end, or proximate the irrigation channel 320), at any suitable location along an irrigation conduit, or at any suitable component in fluid communication with the surgical site (e.g., within in a separate tube or probe).

Controller 360 is a computer that controls parts of fluidics subsystem 110, such as valves or pumps, in response to pressure changes to maintain a target pressure at the surgical site. Controller 360 may determine the IOP from a pressure associated with a surgical site of the eye, or “surgical site pressure”, which may be measured at the surgical site or elsewhere. Surgical system 10 may have one or more sensors at different locations that measure the surgical site pressure. For example, a sensor may be located at or inside of the eye to directly measure the IOP of the eye. As another example, the irrigation pressure measured at an irrigation conduit and/or the aspiration pressure measured at an aspiration conduit may indicate the IOP. The surgical site pressure may not be the same as the IOP, but may correspond to the IOP in that a higher surgical site pressure indicates a higher IOP and a lower surgical site pressure indicates a lower IOP. The surgical site pressure may have a target range that corresponds to the target IOP of the eye, e.g., a value in the range of 0 to 110 mmHg (e.g., a value in the range of 10 to 30, 30 to 55, 55 to 80, and/or 80 to 100 mmHg, such as 30 to 80 mmHg). For example, the irrigation pressure may have a target range of 0 to 110 mmHg (e.g., a value in the range of 0 to 30, 30 to 70, or 70 to 110 mmHg), or the aspiration pressure may have a target range of −760 to 110 mmHg (e.g., a value in the range of −760 to −300, −300 to −100, or −100 to 110 mmHg).

Controller 360 may retrieve one or more pressure thresholds from a memory. In response to detecting that a pressure has reached a pressure threshold, controller 360 provides instructions to bring the pressure back to the target range. For example, to mitigate a post-occlusion break volume surge, a first pressure threshold may indicate when the surgical site pressure has decreased to an unacceptable threshold in response to an occlusion breakage, e.g., when an undesirable volume demand has been created. In response, controller 360 decreases the vacuum pressure in aspiration conduit 303 and/or irrigation conduit 302 to mitigate the rapid decrease of the surgical site pressure. For example, controller 360 may provide fluid to meet the undesirable volume demand to bring the surgical site pressure closer to a desirable level. The first pressure threshold may have any suitable value, e.g., a value in the range of 0 to 207 mmHg (e.g., a value in the range of 0 to 35, 35 to 100, or 100 to 207 mmHg).

A second pressure threshold may indicate when the surgical site pressure is at an acceptable threshold, indicating the surgical site pressure has recovered. In response, controller 360 ceases decreasing the vacuum pressure in the aspiration conduit 303 and/or irrigation conduit 302. The second pressure threshold may have any suitable value, e.g., a value in the range of −760 to 207 mmHg (e.g., a value in the range of −760 to −600, −600 to −400, −400 to −200, −200 to 0, or 0 to 207 mmHg). In some embodiments, the second pressure threshold may be selected such that controller 360 stops decreasing the vacuum pressure before the target IOP range is reached, since the vacuum pressure typically continues to decrease for a short while after controller 360 acts to stop the decrease.

While the above example uses a first pressure threshold defined in terms of an irrigation pressure and a second pressure threshold defined in terms of an aspiration pressure, the first and second thresholds may be defined in terms of use suitable type of pressure (e.g., aspiration pressure, an irrigation pressure, or an intraocular pressure) from any suitable sensors that indicate the pressure at the surgical site. In addition, the first and/or second thresholds can be defined in terms of the same or different types of pressure, e.g., both thresholds could be defined in terms of an aspiration pressure.

### Variable Volume Chamber

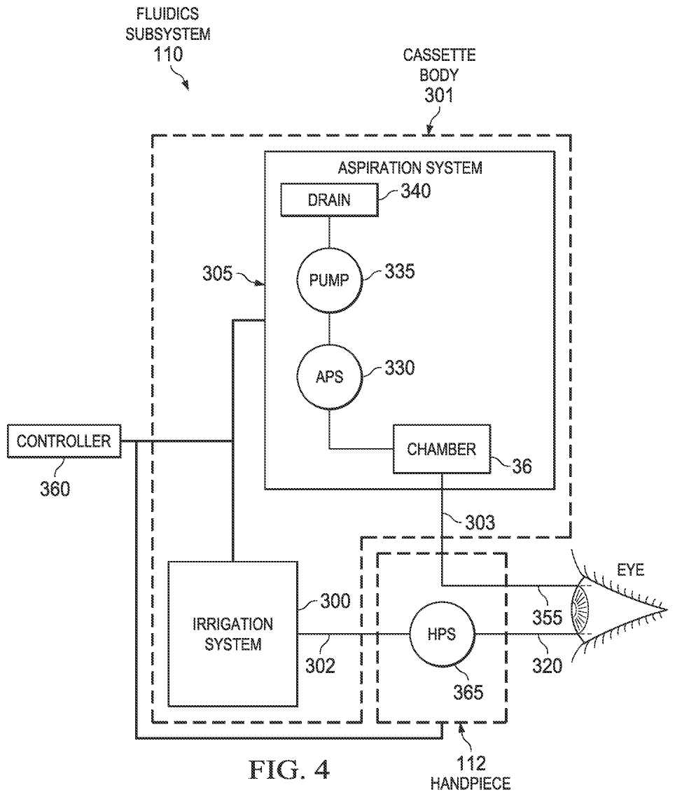

FIG. 4 illustrates an example of fluidics subsystem 110 of FIG. 3 that includes an example of a variable volume chamber 36 that may mitigate post-occlusion break surges.

In the illustrated embodiment, fluidics subsystem 110 includes irrigation system 300 and aspiration system 305 that manage fluid for handpiece 112. Aspiration system 305 includes an aspiration conduit 303, a chamber 36, an aspiration pressure sensor (APS) 330, an aspiration pump 335, and a drain reservoir 340 in fluid communication along aspiration paths as shown. APS 330 detects the aspiration pressure within aspiration conduit 303. Aspiration pump 335 creates a vacuum pressure within aspiration conduit 303 between pump 335 and the eye to draw fluid from the surgical site and into the drain reservoir 340. In certain embodiments, aspiration pump 335 may be reversed to supply fluid to aspiration conduit 303 to mitigate a post-occlusion break surge. Drain reservoir 340 receives the fluid from the surgical site.

Chamber 36 is a variable volume (or collapsible) chamber that expands to store fluid and collapses to move the fluid to meet a volume demand in aspiration conduit 303 to mitigate a post-occlusion break surge. “Expanding” refers to becoming larger in volume, and “collapsing” refers to becoming smaller in volume. Chamber 36 may expand and collapse in any suitable manner. In certain embodiments, chamber 36 comprises a piston that moves in a first direction to expand chamber 36 to store the fluid, and moves in a second direction to collapse chamber 36 to move the fluid. The piston may have any suitable size and shape to fit closely within at least part of chamber 36 to form a fluid-tight seal. For example, the piston may have a cylindrical shape (or other shape) to fit within a cylindrical tube (or other correspondingly shaped tube) of chamber 36. As the piston moves in a first direction, the volume of chamber 36 increases. As the piston moves in a second direction (typically the opposite direction of the first direction), the volume of chamber 36 decreases.

In certain embodiments, chamber 36 comprises a membrane that changes to a first shape to expand chamber 36 to store the fluid, and changes to a second shape to collapse chamber 36 to move the fluid. The membrane may be a flexible, optionally stretchable, sheet material that forms at least a part of chamber 36, and may have any suitable size and shape to store the fluid in chamber 36. The first shape of the membrane may be a shape at which the membrane holds a larger (or even maximum) volume of fluid (e.g., a spherical shape), and the second shape of the membrane may be a shape at which the membrane holds a smaller (or even minimum) volume of fluid.

In certain embodiments, chamber 36 comprises a foldable portion that unfolds to expand chamber 36 to store the fluid, and folds to collapse chamber 36 to move the fluid. The foldable portion may be a semi-rigid sheet material with one or more folds that forms at least a part of chamber 36, and may have any suitable size and shape to store the fluid in chamber 36. At least one fold may unfold to increase the volume of chamber 36, and at least one fold may fold to reduce the volume of chamber 36. As an example, the foldable portion may be shaped like an accordion.

Chamber 36 may have any suitable component that facilitates the expansion and/or collapsing of chamber 36. For example, chamber 36 may have a valve (e.g., a solenoid valve) that expands chamber 36 to store the fluid, and collapses chamber 36 to move the fluid.

Chamber 36 may expand and/or collapse actively (e.g., in response to instructions from controller 36) or passively. In active embodiments, controller 360 may instruct chamber 36 to collapse in response to the pressure detected by one or more pressure sensors. In passive embodiments, chamber 36 may be configured to collapse to move the fluid to meet the volume demand in the aspiration conduit absent instructions from controller 360. For example, chamber 36 may have a pressure-sensitive actuator that collapses chamber 36 in response to a change in pressure from a post-occlusion break surge.

FIG. 5 illustrates an example of a handpiece 112 of FIG. 3 that includes a variable volume chamber 36 that may mitigate post-occlusion break surges. Chamber 36 is a variable volume chamber that expands to store fluid and collapses to move the fluid to meet the volume demand in aspiration conduit 303 to mitigate a post-occlusion break surge. Chamber 36 may be as described with respect to FIG. 4.

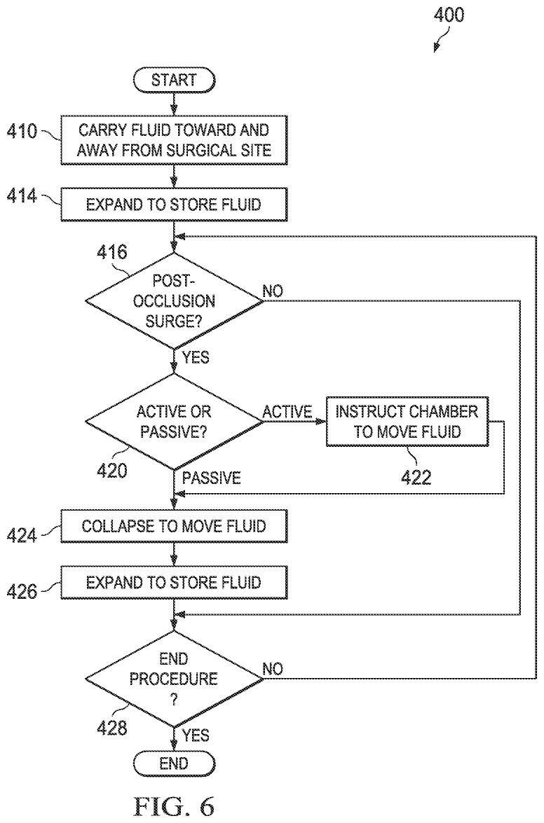

FIG. 6 illustrates an example of a method 400 that may be used by fluidics subsystem 110 of FIG. 3 to mitigate a post-occlusion break surge during a surgical procedure, according to certain embodiments. The method starts at step 410, where an irrigation system carries fluid toward a surgical site, and an aspiration system carries fluid away from the surgical site.

A chamber of the aspiration system expands to store fluid at step 414. In certain embodiments, the chamber comprises a piston that moves in a first direction to expand the chamber. In other embodiments, the chamber comprises a membrane that changes to a first shape to expand the chamber. In yet other embodiments, the chamber comprises a foldable portion that unfolds to expand the chamber.

A post-occlusion break surge may occur at step 416. If a post-occlusion break surge occurs, the method proceeds to step 420, where the mitigation process may be active or passive. If the process is active, the method proceeds to step 422, where a controller instructs the chamber to move the fluid. The method then proceeds to step 424. If the process is passive, the method proceeds directly to step 424. If a post-occlusion break surge does not occur, the method proceeds to step 428.

The chamber collapses to move the fluid to meet the volume demand at step 424 to mitigate the post-occlusion break surge. In certain embodiments, the chamber comprises a piston that moves in a second direction (e.g., a direction opposite to the first direction of step 414) to collapse the chamber. In other embodiments, the chamber comprises a membrane that changes to a second shape to collapse the chamber. In yet other embodiments, the chamber comprises a foldable portion that folds to collapse the chamber.

The chamber expands to store fluid at step 426 to prepare for another post-occlusion break surge. The chamber may expand in a manner as described in step 414, and may expand actively or passively. The procedure may end at step 428. If the procedure is not ending, the method returns to step 416. If the procedure is ending, the method ends.

### Chamber System

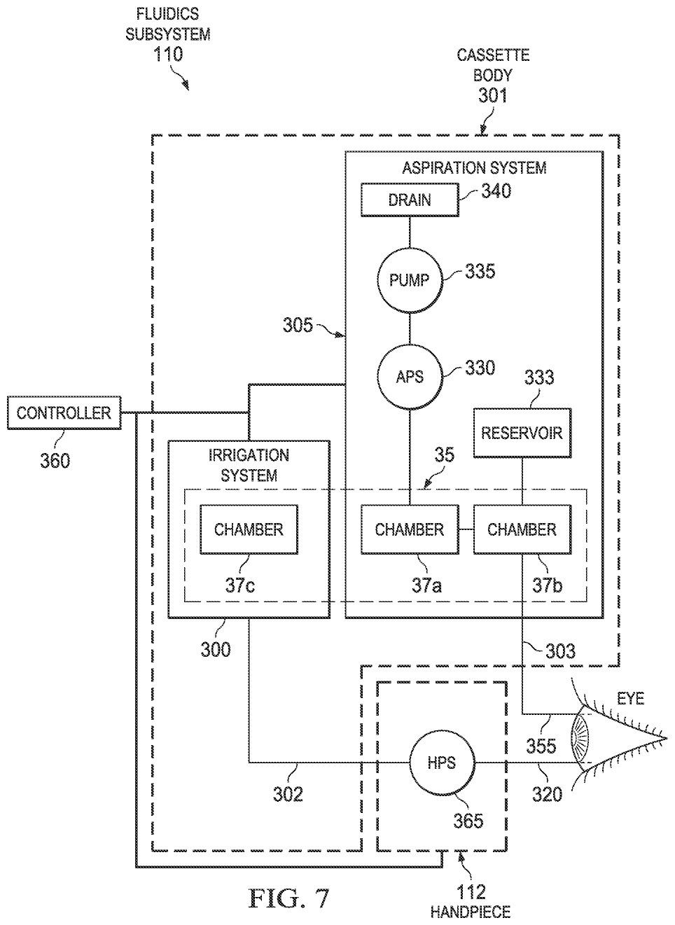

FIG. 7 illustrates an example of fluidics subsystem 110 of FIG. 3 that includes an example of a chamber system 35 that may mitigate post-occlusion break surges. In the illustrated embodiment, fluidics subsystem 110 includes irrigation system 300 and aspiration system 305 that manage fluid for handpiece 112. Aspiration system 305 includes an aspiration conduit 303, a chamber system 37 (37a-c), an aspiration pressure sensor (APS) 330, an aspiration pump 335, a drain reservoir 340, and a reservoir 333 in fluid communication along aspiration paths as shown. APS 330 detects the aspiration pressure within aspiration conduit 303. Aspiration pump 335 creates a vacuum pressure within aspiration conduit 303 between pump 335 and the eye to draw fluid from the surgical site and into drain reservoir 340. In certain embodiments, aspiration pump 335 may be reversed to supply fluid to aspiration conduit 303 to mitigate a post-occlusion break surge. Drain reservoir 340 receives the fluid from the surgical site. Reservoir 333 stores fluid that may be used for surge mitigation, and may be implemented as one or more reservoirs. Examples of reservoir 333 include a venturi, drain, vent, irrigation, and other suitable reservoir.

Chamber system 35 comprises a plurality of chambers 37 (37a-c). Chamber system 35 stores fluid in chambers 37, and meets a volume demand in the aspiration conduit by moving fluid from one or more of the chambers 37 to mitigate a post-occlusion break surge. A chamber 37 may be at any suitable location. For example, a chamber 37a may be disposed along a drain path leading to drain reservoir 340. As another example, a chamber 37b may be disposed along a reservoir path leading to reservoir 333. As another example, a chamber 37c may be disposed along an irrigation conduit 302 of irrigation system 300.

In certain embodiments, chamber system 35 may meet the volume demand by moving fluid. Chamber system 35 may move fluid based on a characteristic of the post-occlusion break surge. Examples of such movement include: (1) Chamber system 35 may move fluid from more chambers 37 for a larger post-occlusion break surge, and move fluid from fewer chambers 37 for a smaller post-occlusion break surge. (2) Chamber system 35 may move fluid from one or more larger chambers 37 for a larger post-occlusion break surge, and move fluid from one or more smaller chambers 37 for a smaller post-occlusion break surge. (3) A chamber 37 closer to an increase in pressure related to a surge may move fluid before or instead of a chamber 37 farther away from the pressure increase.

In certain embodiments, chamber system 35 may meet the volume demand actively or passively by moving fluid actively or passively. Chamber system 35 may move fluid actively (e.g., in response to instructions from controller 36) or passively. In certain active embodiments, controller 36 may determine a size and/or location of a post-occlusion break surge, and then instruct chamber system 35 to respond as described above. In certain passive embodiments, a chamber 37 may have a pressure-sensitive component, such that the chamber 37 moves fluid as described above. For example, a chamber 37 closer to an increase in surge-related pressure may move fluid before a chamber 37 farther away. As another example, more chambers 37 may move fluid for a larger increase in surge-related pressure than a smaller increase.

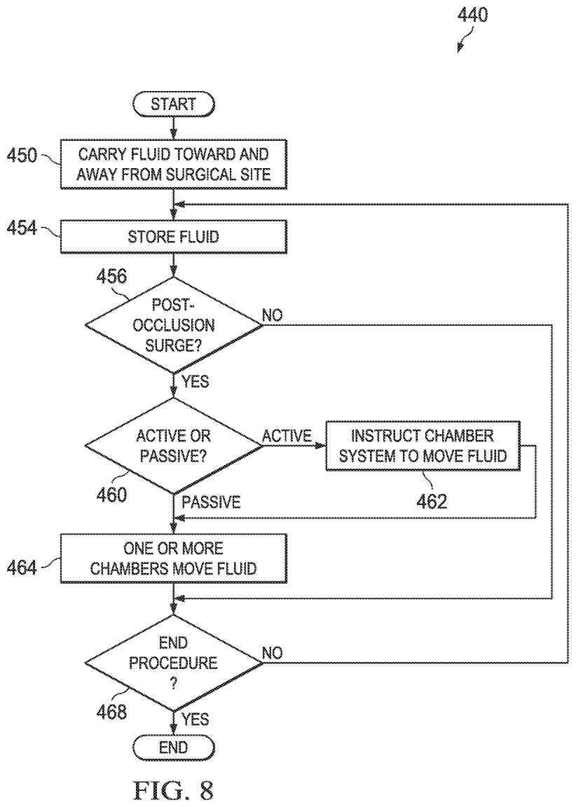

FIG. 8 illustrates an example of a method 440 that may be used by fluidics subsystem 110 of FIG. 3 to mitigate a post-occlusion break surge during a surgical procedure, according to certain embodiments. The method starts at step 450, where an irrigation system carries fluid toward a surgical site, and an aspiration system carries fluid away from the surgical site.

The chambers of a chamber system of the aspiration system store fluid at step 454. In certain embodiments, the chamber may receive fluid provided by a fluid source and/or may expand to store the fluid.

A post-occlusion break surge may occur at step 456. If a post-occlusion break surge occurs, the method proceeds to step 460, where the mitigation process may be active or passive. If the process is active, the method proceeds to step 462, where a controller instructs the chamber system to move the fluid. The controller may determine characteristics of the surge, and then move the fluid in accordance with the characteristics. The method then proceeds to step 464. If the process is passive, the method proceeds directly to step 464. If a post-occlusion break surge does not occur at step 456, the method proceeds to step 468.

The chamber system moves fluid at step 464 to mitigate the post-occlusion break surge. The way the fluid is moved may be based on characteristics of the surge. The procedure may end at step 468. If the procedure is not ending, the method returns to step 454, where the chambers store fluid to prepare to mitigate the next surge. If the procedure is ending, the method ends.

### Priming Procedures

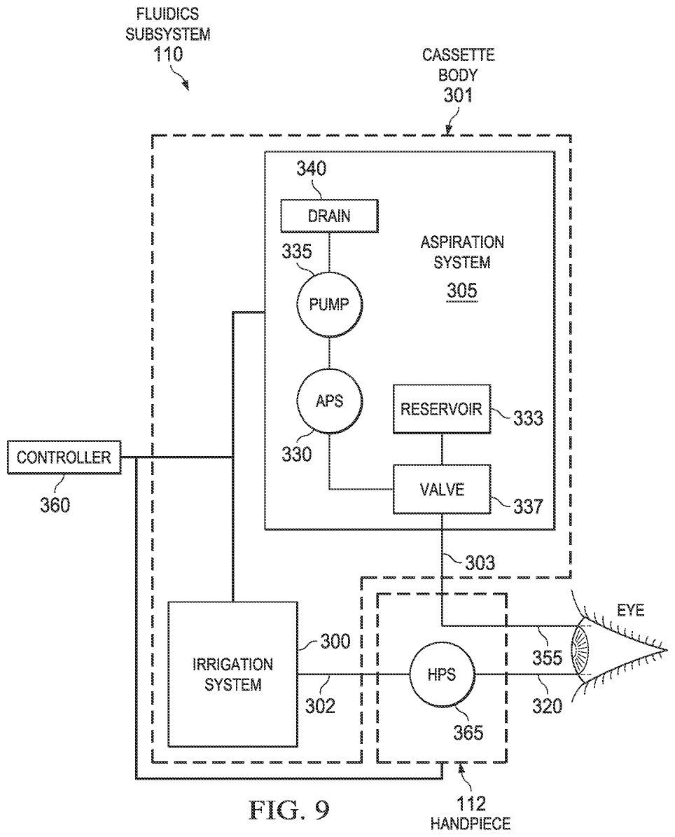

FIG. 9 illustrates an example of fluidics subsystem 110 of FIG. 3 that performs a priming procedure that may improve mitigation of post-occlusion break surges. In general, air bubbles trapped in the vacuum path collapse during surge mitigation, which may compromise the effectiveness and predictability of the mitigation. In the priming procedure, aspiration flow is vibrated back and forth to reduce air bubbles trapped in the vacuum path, which may improve surge mitigation. In certain embodiments, controller 360 sends instructions to perform the priming procedure. The instructions may be sent automatically (e.g., prior to a procedure) or in response to user input.

In the illustrated embodiment, fluidics subsystem 110 includes irrigation system 300 and aspiration system 305 that manage fluid for handpiece 112. Aspiration system 305 includes an aspiration conduit 303, an aspiration pressure sensor (APS) 330, an aspiration pump 335, a drain reservoir 340, a reservoir 333, and a valve 377 in fluid communication along aspiration paths as shown. APS 330 detects the aspiration pressure within aspiration conduit 303. Aspiration pump 335 creates a vacuum pressure within aspiration conduit 303 between pump 335 and the eye to draw fluid from the surgical site and into drain reservoir 340. Drain reservoir 340 receives the fluid from the surgical site.

Reservoir 333 stores fluid that may be used for surge mitigation, and may be implemented as one or more reservoirs. Examples of reservoir 333 include a venturi, drain, vent, irrigation, and other suitable reservoir. Valve 337 manages fluid of aspiration conduit 303 to perform a priming procedure. Valve 377 may be implemented as one or more valves. “Opening” a valve may refer to opening a valve of multiple valves, and “closing” a valve may refer to closing the same valve or another valve of the multiple valves. In certain embodiments, controller 360 sends instructions to valve 337 to perform the priming procedure.

The priming procedure moves, e.g., vibrates, the fluid within aspiration conduit 303 back and forth to reduce air (e.g., air bubbles) within aspiration conduit 303. The procedure may vibrate the fluid with any suitable frequency, e.g., in a range from low frequencies to ultrasound frequencies, such as 1 vibration per second to 70 kHz. The movement may be produced in any suitable manner. Examples of techniques for producing movement include: (1) Valve 337 may rapidly switch between opening to move fluid into aspiration conduit 303 and closing to cease the move of fluid, causing the fluid to vibrate. (2) Aspiration pump 335 may rapidly change the vacuum pressure within aspiration conduit 303 to cause the fluid to vibrate. (3) Aspiration pump 335 may change the vacuum pressure and valve 337 may move fluid in a coordinated manner to vibrate the fluid. (4) Cassette body 301 may be mechanically vibrated using the fluidics mechanism located at console 100. (5) Tubing 302 or 303 itself may be mechanically vibrated. (6) A phaco handpiece 112 may be run during priming. In certain embodiments, controller 360 sends instructions to the related component to perform the priming procedure.

In certain embodiments, aspiration conduit 303 may comprise tubing that facilitates removal of air bubbles. In the embodiments, the tubing may have an internal surface where air bubbles are not likely to adhere. For example, the surface may be hydrophilic or superhydrophilic (e.g., very low water contact angles of less than 10°), which allows for dispersion of water droplets in seconds. Examples of such tubing are superhydrophilic polydimethylsiloxane (PDMS) tubes.

### Irrigation System Mitigation

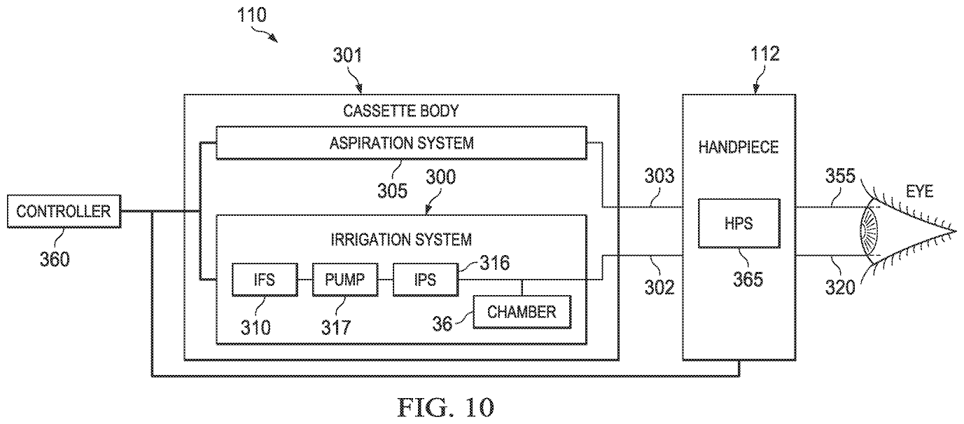

FIG. 10 illustrates an example of fluidics subsystem 110 of FIG. 3 that includes an example of an irrigation system 300 that may mitigate post-occlusion break surges. In the embodiment, irrigation system 300 carries fluid toward the surgical site via an irrigation conduit. In response to a post-occlusion break surge, irrigation system 300 moves additional fluid to meet a volume demand in the irrigation conduit to mitigate the post-occlusion break surge.

In the illustrated embodiment, fluidics subsystem 110 includes irrigation system 300 and aspiration system 305 that manage fluid for handpiece 112. Irrigation system 300 includes an irrigation conduit 302, an irrigation fluid source (IFS) 310, an irrigation pump 317, an irrigation pressure sensor (IPS) 316, and a chamber 36 in fluid communication along irrigation paths as shown. IPS 316 measures pressure within irrigation conduit 302. IFS 310 supplies irrigation fluid, e.g., a sterile saline fluid. Irrigation pump 317 generates pressure to provide fluid from IFS 310 to irrigation conduit 302.

Chamber 36 stores fluid and moves the fluid to meet the volume demand in irrigation conduit 302. In certain embodiments, chamber 36 is a variable volume chamber (as described with respect to FIGS. 4 through 6) that expands to store fluid and collapses to move the fluid. In other embodiments, chamber 36 represents a chamber system of irrigation system 300 (as described with respect to FIGS. 7 and 8) stores fluid and moves fluid from one or more chambers of the chamber system. In certain embodiments, controller 360 sends instructions to irrigation system 300 to mitigate a post-occlusion break surge.

In certain embodiments, in response to a change of vacuum pressure indicating an occlusion or a surge, irrigation system 300 moves additional fluid (such as pressurized fluid) into irrigation conduit 302 to mitigate the post-occlusion break surge. Irrigation system 300 may move the additional fluid in any suitable manner, including as in one or more of the following examples: (1) Irrigation pump 317 increases pressure to provide additional fluid from IFS 310 to irrigation conduit 302. (2) Chamber 36 is a variable volume chamber that expands to store additional fluid and collapses to move the additional fluid into irrigation conduit 302. (2) Chamber 36 is a chamber system that moves the additional fluid from one or more chambers of the chamber system into irrigation conduit 302. (3) A valve or other fluid switch may move additional fluid stored at any suitable fluid source at any suitable location of the irrigation path. The stored additional fluid may be pressurized fluid from an air pressure supply, irrigation bag chamber, peristaltic pump, and/or compliant chamber in the irrigation path.

The movement of fluid may be active or passive. In active embodiments, controller 360 detects that pressure has reached a threshold and instructs a component of irrigation system 300 (e.g., irrigation pump 317, chamber 36, or a valve) to move additional fluid into irrigation conduit 302. In passive embodiments, in response to pressure reaching a threshold, a component of irrigation system 300 moves fluid into irrigation conduit 302.

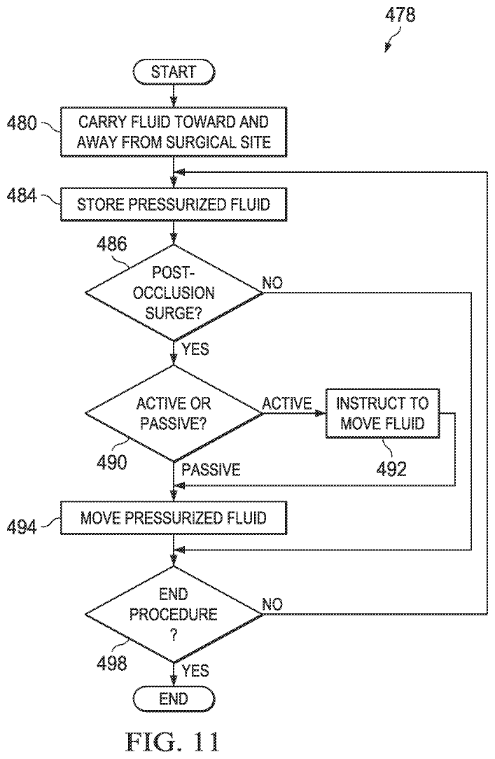

FIG. 11 illustrates an example of a method 478 that may be used by fluidics subsystem 110 of FIG. 3 to mitigate a post-occlusion break surge during a surgical procedure, according to certain embodiments. The method starts at step 480, where an irrigation system carries fluid toward a surgical site, and an aspiration system carries fluid away from the surgical site.

The irrigation system stores additional fluid at step 484. The additional fluid may be stored at, e.g., a chamber (e.g., a variable volume chamber or a chamber system), an irrigation fluid source (IFS), or other suitable fluid source. In certain embodiments, the fluid may be pressurized. A post-occlusion break surge may occur at step 486. If a post-occlusion break surge occurs, the method proceeds to step 490, where the mitigation process may be active or passive. If the process is active, the method proceeds to step 492, where a controller instructs the irrigation system to move the stored additional fluid. The method then proceeds to step 494. If the process is passive, the method proceeds directly to step 494. If a post-occlusion break surge does not occur at step 486, the method proceeds to step 498.

The irrigation system moves the additional fluid at step 494 to mitigate the post-occlusion break surge. The procedure may end at step 498. If the procedure is not ending, the method returns to step 484, where the irrigation system stores fluid to prepare to mitigate the next surge. If the procedure is ending, the method ends.

### Surge Mitigating Tubing

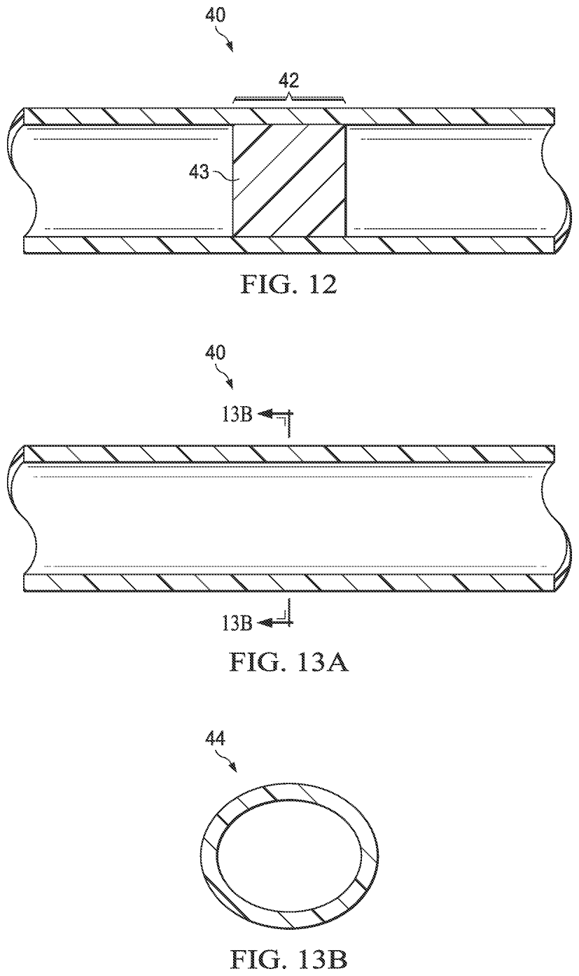

FIGS. 12 to 14C illustrate examples of tubing 40 of aspiration system 305 of fluidics subsystem 110 of FIG. 3 that may mitigate a post-occlusion break surge during a surgical procedure. In certain embodiments, an aspiration pump generates a normal vacuum within an aspiration conduit during normal operation to carry fluid away from the surgical site. The aspiration pressure may have a target range of −760 to 0 mmHg, as described with reference to FIG. 3. Tubing 40 has a larger cross-sectional area during normal aspiration flow and vacuum than under occlusion.

An occlusion increases the vacuum within the aspiration conduit to an occlusion vacuum limit. The occlusion vacuum limit may have a range of 0 to 760 mmHg. In response to the occlusion vacuum of a particular duration (e.g., greater than 10 milliseconds (ms)), tubing 40 collapses from the larger cross-sectional area to a smaller cross-sectional area. The occlusion breaks through, which creates a post-occlusion break surge. Tubing 40 maintains the smaller cross-sectional area during the duration of the surge (and perhaps longer) to resist the surge. Typically, a surge lasts up to 500 ms, but may last up to 1 second (s). Tubing 40 returns to the larger cross-sectional area after the surge duration.

Tubing 40 may have a mitigating viscoelastic material and/or cross-section that allows tubing 40 to collapse and maintain the smaller cross-sectional area to mitigate the surge. The mitigating viscoelastic material and/or cross-section may be present throughout tubing 40 or in one or more mitigating sections of tubing 40. A mitigating section may have any suitable length, such as between 0.1 to 10 centimeters (cm) (e.g., a value in the range of 0.1 to 1, 1 to 2, 2 to 5, to 7, and/or 7 to 10 cm). Tubing 40 may have one or more mitigating sections, where each section has a mitigating viscoelastic material or a mitigating cross-section or both. Examples of such tubing are described with reference to FIGS. 12 to 14B.

FIG. 12 illustrates an example of tubing 40 that comprises a mitigating viscoelastic material. In the example, tubing 40 has a mitigating section 42 comprising a viscoelastic material 43. Examples of such materials include low durometer polyvinylchloride, silicone elastomers, polyurethane, or high density polyethylene.

FIGS. 13A and 13B illustrate an example of tubing 40 with a mitigating cross-section 44. Cross-section 44 may have an elliptical (including circular), marquise, polygonal, x-shaped, or any other suitable shape. In the example, cross-section 44 is elliptical.

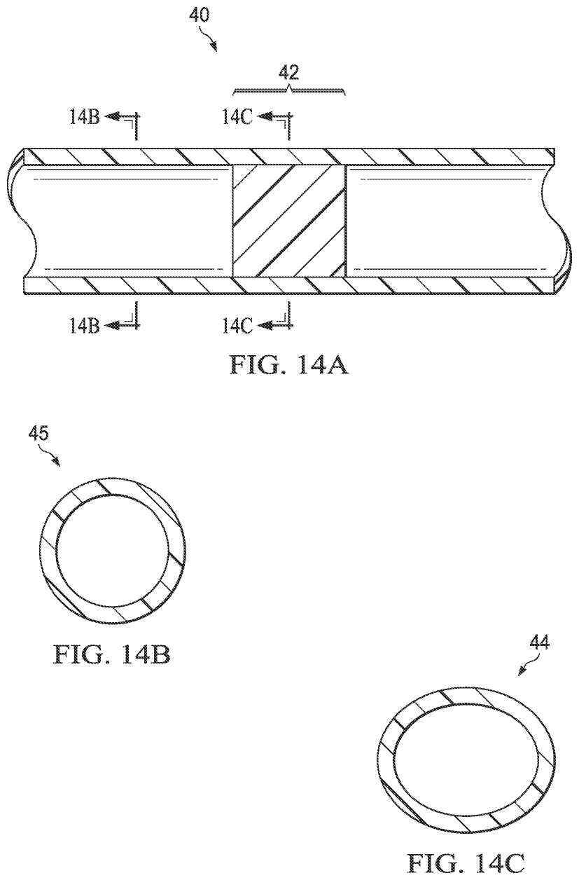

FIGS. 14A through 14C illustrate an example of tubing 40 with a mitigating section 42 with a mitigating cross-section 44. In the example, mitigating cross-section 44 of mitigating section 42 is elliptical, and the cross section 45 of the rest of tubing 40 is circular.

A component (such as the computer 103 or controller 360) of the systems and apparatuses disclosed herein may include an interface, logic, and/or memory, any of which may include hardware and/or software. An interface can receive input to the component, send output from the component, and/or process the input and/or output. Logic can perform operations of the component. Logic may include one or more electronic devices that process data, e.g., execute instructions to generate output from input. Examples of such an electronic device include a computer, processor or microprocessor (e.g., a Central Processing Unit (CPU), and computer chip. Logic may include computer software that encodes instructions capable of being executed by the electronic device to perform operations. Examples of computer software includes a computer program, an application, and an operating system.

A memory can store information and may comprise tangible, computer-readable, and/or computer-executable storage medium. Examples of memory include computer memory (e.g., Random Access Memory (RAM) or Read Only Memory (ROM)), mass storage media (e.g., a hard disk), removable storage media (e.g., a Compact Disk (CD) or Digital Video or Versatile Disk (DVD)), database and/or network storage (e.g., a server), and/or other computer-readable media. Particular embodiments may be directed to memory encoded with computer software.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, or the operations of the systems and apparatuses may be performed by more, fewer, or other components. As another example, modifications may be made to the methods disclosed herein. The methods may include more, fewer, or other steps, and the steps may be performed in any suitable order.

Regarding the example embodiments, parts described as internal to a component may be distributed external to the component. In certain embodiments, parts of an aspiration system and/or irrigation system may be external to a cassette body. In certain embodiments, parts of a pump of an aspiration system in a cassette body may be external to the cassette body. For example, a motor that drives the pump may be located at a console. In certain embodiments, parts of a sensor of an aspiration system in a cassette body may be external to the cassette body. For example, a processor that receives sensor readings may be located at a console.

To aid the Patent Office and readers in interpreting the claims, Applicants wish to note that they do not intend any of the claims or claim elements to invoke 35 U.S.C. § 112(f) unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the applicants to refer to structures known to those skilled in the relevant art and is not intended to invoke 35 U.S.C. § 112(f).

What is claimed:

1. A surgical cassette for an ophthalmic surgical system, comprising:
an irrigation system in fluid communication with a handpiece and configured to carry fluid toward a surgical site;
an aspiration system in fluid communication with the handpiece via an aspiration conduit, the aspiration system configured to carry fluid away from the surgical site; and
a computer configured to instruct the aspiration system to vibrate fluid back and forth within the aspiration conduit to perform a priming procedure.

2. The surgical cassette of claim 1, wherein the aspiration system comprises a valve configured to vibrate fluid by:
opening to move fluid into the aspiration conduit; and
closing to cease moving the fluid into the aspiration conduit.

3. The surgical cassette of claim 1, wherein the aspiration system comprises an aspiration pump configured to:
create a vacuum pressure within the aspiration conduit; and
vibrate fluid by changing the vacuum pressure within the aspiration conduit.

4. The surgical cassette of claim 1, wherein the aspiration conduit comprises tubing with an internal surface that is hydrophilic to facilitate removal of air bubbles within the tubing.

5. The surgical cassette of claim 1, wherein the aspiration conduit comprises tubing with an internal surface that is superhydrophilic to facilitate removal of air bubbles within the tubing.

6. The surgical cassette of claim 1, wherein the aspiration system comprises:
a fluidics mechanism configured to vibrate the surgical cassette to vibrate the fluid.

7. The surgical cassette of claim 1, wherein the aspiration system comprises:
tubing configured to carry the fluid; and
a fluidics mechanism configured to vibrate the tubing.

8. The surgical cassette of claim 1, wherein the computer is configured to instruct the handpiece to run to vibrate the fluid.

9. A surgical cassette for an ophthalmic surgical system, comprising:
an irrigation system in fluid communication with a handpiece and configured to carry fluid toward a surgical site;
an aspiration system in fluid communication with the handpiece via an aspiration conduit, the aspiration system configured to carry fluid away from the surgical site; and
a computer configured to instruct the handpiece to run to vibrate fluid back and forth within the aspiration conduit to perform a priming procedure.

10. The surgical cassette of claim 9, wherein the aspiration conduit comprises tubing with an internal surface that is hydrophilic to facilitate removal of air bubbles within the tubing.

11. The surgical cassette of claim 9, wherein the aspiration conduit comprises tubing with an internal surface that is superhydrophilic to facilitate removal of air bubbles within the tubing.

12. The surgical cassette of claim 9, the computer configured to instruct the aspiration system to vibrate fluid back and forth within the aspiration conduit.

13. The surgical cassette of claim 12, wherein the aspiration system comprises:
a fluidics mechanism configured to vibrate the surgical cassette to vibrate the fluid.

14. The surgical cassette of claim 12, wherein the aspiration system comprises:
tubing configured to carry the fluid; and
a fluidics mechanism configured to vibrate the tubing.

15. A method for performing a priming procedure for a surgical cassette, comprising:
carrying, by an irrigation system in fluid communication with a handpiece, fluid toward a surgical site;
carrying, by an aspiration system in fluid communication with the handpiece via an aspiration conduit, fluid away from the surgical site; and
instructing, by a computer, the aspiration system to vibrate fluid back and forth within the aspiration conduit.

16. The method of claim 15, further comprising:
opening, by a valve of the aspiration system, to move fluid into the aspiration conduit; and
closing, by the valve, to cease moving the fluid into the aspiration conduit.

17. The method of claim 15, further comprising:
creating, by an aspiration pump of the aspiration system, a vacuum pressure within the aspiration conduit; and
vibrating, by the aspiration pump, fluid by changing the vacuum pressure within the aspiration conduit.

18. The method of claim 15, further comprising:
vibrating, by a fluidics mechanism of the aspiration system, the surgical cassette to vibrate the fluid.

19. The method of claim 15, further comprising:
vibrating, by a fluidics mechanism of the aspiration system, tubing of the aspiration system configured to carry the fluid to vibrate the fluid.

20. A surgical cassette for an ophthalmic surgical system, comprising:
an irrigation system in fluid communication with a handpiece and configured to carry fluid toward a surgical site;
an aspiration system in fluid communication with the handpiece via an aspiration conduit, the aspiration system configured to carry fluid away from the surgical site; and
a computer configured to:
instruct the aspiration system to vibrate fluid back and forth within the aspiration conduit to perform a priming procedure; and
instruct the handpiece to run to vibrate fluid;
the aspiration system comprising:
a valve configured to vibrate fluid by: opening to move fluid into the aspiration conduit, and closing to cease moving the fluid into the aspiration conduit;
an aspiration pump configured to: create a vacuum pressure within the aspiration conduit, and vibrate fluid by changing the vacuum pressure within the aspiration conduit;
tubing with an internal surface that is hydrophilic to facilitate removal of air bubbles within the tubing; and
a fluidics mechanism configured to vibrate the surgical cassette and the tubing to vibrate the fluid.
